(19) United States
(12) Patent Application Publication
(10) Pub. No.: US 2026/0049353 A1
(43) Pub. Date: Feb. 19, 2026

(54) MULTIPLEXED RNA QUANTIFICATION USING CRISPR-CAS13

(71) Applicant: The Trustees of Princeton University, Princeton, NJ (US)

(72) Inventors: Brian KANG, Princeton, NJ (US); Alexander Ploss, Princeton, NJ (US); Cameron Myhrvold, Princeton, NJ (US)

(73) Assignee: The Trustees of Princeton University, Princeton, NJ (US)

(21) Appl. No.: 19/302,644

(22) Filed: Aug. 18, 2025

Related U.S. Application Data
(60) Provisional application No. 63/683,774, filed on Aug. 16, 2024.

(51) Int. Cl. C12Q 1/686; C12N 9/22; C12N 15/113; C12Q 1/48; C12Q 1/6851; C12Q 1/6876; G16B 30/00; G16B 40/20; G16H 50/30

(52) U.S. Cl. CPC C12Q 1/686; C12N 9/226; C12N 15/113; C12Q 1/48; C12Q 1/6851; C12Q 1/6876; C12Y 207/07006; G16B 30/00; G16B 40/20; G16H 50/30; C12N 2310/20; C12Q 2600/16

(57) ABSTRACT

The present disclosure provides a method for multiplexed RNA quantification, comprising: initiating an enzymatic reaction by mixing a reaction mixture, the reaction mixture comprising a CRISPR effector protein, a target-specific crRNA, a fluorescent reporter molecule, T7 RNA polymerase, an input sample, and a reaction buffer; capturing a plurality of fluorescence measurements, each fluorescence measurement captured at a different point in time; and determining a plurality of relative target concentrations by fitting the plurality of fluorescence measurements to a mathematical model of the enzymatic reaction. The method enables highly multiplexed quantification of RNA targets using CRISPR-based detection.

### CROSS-REFERENCE TO RELATED APPLICATIONS

This application claims priority to U.S. Provisional Patent Application No. 63/683,774, filed Aug. 16, 2024, titled MULTIPLEXED RNA QUANTIFICATION, filed Aug. 16, 2024, which is hereby incorporated by reference in its entirety.

### STATEMENT REGARDING FEDERALLY SPONSORED RESEARCH OR DEVELOPMENT

This invention was made with government support under Grant Nos. AI138797, AI153236, AI146917, AI168048, AI107301, AI168808 and UL1TR003017 awarded by the National Institutes of Health and Grant No. 75D30122C15113 awarded by the Centers for Disease Control. The government has certain rights in the invention.

### FIELD OF INVENTION

The present disclosure relates to methods and systems for RNA quantification, and more particularly to a highly multiplexed RNA quantification technique using CRISPR-Cas13 and microfluidics.

### BACKGROUND

RNA quantification tools play a crucial role in understanding gene expression patterns and cellular responses to various stimuli. Traditional methods for RNA quantification include quantitative reverse transcription polymerase chain reaction (RT-qPCR) and RNA sequencing (RNA-seq). While these techniques have been widely used, they often face limitations in terms of throughput, cost-effectiveness, or multiplexing capabilities.

RT-qPCR is a sensitive method for quantifying specific RNA targets, but it typically has limited multiplexing capacity and can be labor-intensive when analyzing multiple genes across numerous samples. RNA-seq provides a comprehensive view of the transcriptome but can be expensive for large-scale studies involving many samples or time points.

There is an ongoing need in the field for RNA quantification methods that can bridge the gap between the specificity of RT-qPCR and the high-throughput nature of RNA-seq. Ideally, such methods would allow for the simultaneous analysis of multiple RNA targets across numerous samples while maintaining accuracy and cost-effectiveness.

Recent advances in CRISPR-based technologies have opened up new possibilities for nucleic acid detection and quantification. CRISPR systems, particularly those utilizing Cas13 enzymes, have shown promise for RNA targeting and detection. These systems offer the potential for highly specific and sensitive RNA quantification, but their application to multiplexed, high-throughput RNA analysis has not been fully explored.

Microfluidic technologies have also emerged as powerful tools for miniaturizing and parallelizing biological assays. The integration of microfluidics with RNA quantification methods could potentially enable high-throughput analysis of multiple samples and targets simultaneously.

Despite these technological advances, challenges remain in developing RNA quantification methods that can effectively combine high multiplexing capacity, throughput, sensitivity, and cost-effectiveness. Addressing these challenges could enable new applications in gene expression analysis, including large-scale kinetic studies, gene regulatory network mapping, and comprehensive profiling of cellular responses to various stimuli or perturbations.

### SUMMARY

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

According to an aspect of the present disclosure, a method for multiplexed RNA quantification is provided. The method includes initiating an enzymatic reaction by mixing a reaction mixture, the reaction mixture comprising a CRISPR effector protein, a target-specific crRNA, a fluorescent reporter molecule, T7 RNA polymerase, an input sample, and a reaction buffer. The method further includes capturing a plurality of fluorescence measurements, each fluorescence measurement captured at a different point in time. The method also includes determining a plurality of relative target concentrations by fitting the plurality of fluorescence measurements to a mathematical model of the enzymatic reaction.

According to other aspects of the present disclosure, the method may include one or more of the following features. The CRISPR effector protein may be a Class 2 CRISPR effector protein. The Class 2 CRISPR effector protein may be of Type V. The Class 2 CRISPR effector protein may be of Type VI. The CRISPR effector protein may comprise Cas12, Cas13, LwaCas13a (C2c2), LbCas12a (Cpf1), LbuCas13a, PsmCas13b, PspCas13b, CcaCas13b, AsCas12a, CeCas12a, PbCas12a, or a combination thereof. The method may further comprise determining one or more values quantifying a differential expression across a plurality of treatment conditions. The method may further comprise generating a dataset including the one or more values. The method may further comprise training a machine learning model using the dataset. The machine learning model may be configured to predict a clinical outcome or a disease outcome. The mathematical model may comprise a system of differential equations representing individual reaction components including transcription, cis cleavage, and trans cleavage. Fitting the plurality of fluorescence measurements may include generating a single concentration-associated parameter for each curve. The mixing may occur on a microfluidic chip. Capturing the plurality of fluorescence measurements may include capturing a fluorescence measurement every 1-15 minutes. Capturing the plurality of fluorescence measurements may include capturing fluorescence measurements for 1-6 hours. Capturing the plurality of fluorescence measurements may include capturing fluorescence measurements while the reaction mixture is incubating at a predetermined temperature. The method may further comprise extracting test RNA from a sample. The method may further comprise amplifying a gene in the test RNA in a separate PCR reaction, where the input sample includes a PCR product from the separate PCR reaction. The reaction mixture may consist of the CRISPR effector protein, the target-specific crRNA, the fluorescent reporter molecule, T7 RNA polymerase, the input sample, and the reaction buffer. A plurality of different reaction mixtures may be processed simultaneously. At least 100 different reaction mixtures may be processed simultaneously.

According to another aspect of the present disclosure, an assay module for RNA quantification is provided. The assay module includes a RNase H2-dependent PCR (rhPCR) primer, a crRNA, a Cas13 detection reagent, and a PCR reagent.

According to other aspects of the present disclosure, the assay module may include one or more of the following features. The RNase H2-dependent PCR (rhPCR) primer may comprise a plurality of RNase H2-dependent PCR (rhPCR) primers. The crRNA may comprise a plurality of crRNA. The Cas13 detection reagent may comprise a plurality of Cas13 detection reagents. The PCR reagent may comprise a plurality of PCR reagents.

According to another aspect of the present disclosure, a method for combining RNase H-dependent PCR (rhPCR) amplification and Cas13 detection is provided. The method includes providing an assay module as described above. The method further includes performing RNase H2-dependent multiplexed amplification using a first predetermined concentration of MgCl₂ and a first predetermined enzyme activity of RNase H2 enzyme. The method also includes, in series with the RNase H2-dependent multiplexed amplification, detecting Cas13 using a Cas13 detection reaction having a second predetermined concentration of MgCl₂.

According to other aspects of the present disclosure, the method may include one or more of the following features. The first predetermined concentration may be 1 mM to 5 mM, the first predetermined enzyme activity may be 1 mU/μL to 5 mU/μL, and the second predetermined concentration may be 4 mM to 10 mM.

The foregoing general description of the illustrative embodiments and the following detailed description thereof are merely exemplary aspects of the teachings of this disclosure and are not restrictive.

### BRIEF DESCRIPTION OF FIGURES

Non-limiting and non-exhaustive examples are described with reference to the following figures.

FIG. 1 illustrates a flowchart of a method for multiplexed RNA quantification, according to aspects of the present disclosure.

FIG. 2 is a graph showing the diversity of fluorescence kinetics, using 100× serial dilutions of Huh7.5 RNA extract.

FIG. 3 is a schematic illustration of an embodiment of the disclosed technique; where, following an RNA extraction step, samples are amplified using commercially available rhPCR primers and combined with a Cas13 assay mix in a microfluidic chip. Reactions run for period of time, and fluorescent signals are recorded for downstream analysis.

FIGS. 4A and 4B are plots showing model parameter distributions, and specifically calculated parameter values for GAPDH (4A) and ADAR (4B).

FIGS. 5A and 5B are graphs showing qCARMEN detection of synthetic FGFR2 isoform dilutions, and specifically serial 10× dilutions of IIIb isoform of FGFR2 with IIIc isoform at a constant concentration (5A), and serial 10× dilutions of IIIc isoform with IIIb isoform remaining constant (5B).

FIG. 6 is a graph showing kinetics of EXOC7 isoforms over a 72-hour timecourse.

### DETAILED DESCRIPTION

The following description sets forth exemplary aspects of the present disclosure. It should be recognized, however, that such description is not intended as a limitation on the scope of the present disclosure. Rather, the description also encompasses combinations and modifications to those exemplary aspects described herein.

The present disclosure relates to methods and systems for multiplexed RNA quantification. Multiplexed RNA quantification may allow for simultaneous measurement of expression levels for multiple genes across numerous samples. This approach may enable high-throughput analysis of gene expression changes in response to various stimuli or conditions.

In some cases, multiplexed RNA quantification methods may be applied to study interferon-stimulated gene responses. Interferon-stimulated genes play important roles in immune responses, and analyzing their expression patterns may provide insights into host-pathogen interactions and disease mechanisms.

Multiplexed RNA quantification techniques may also be utilized to examine isoform switching during epithelial-mesenchymal transition. Epithelial-mesenchymal transition involves changes in cell phenotype and gene expression. Quantifying shifts in RNA isoform abundance may reveal details about the molecular processes underlying this cellular transformation.

The methods and systems described herein may offer advantages in terms of throughput, multiplexing capability, and sensitivity compared to traditional RNA quantification approaches. These capabilities may enable new types of large-scale gene expression studies and analyses.

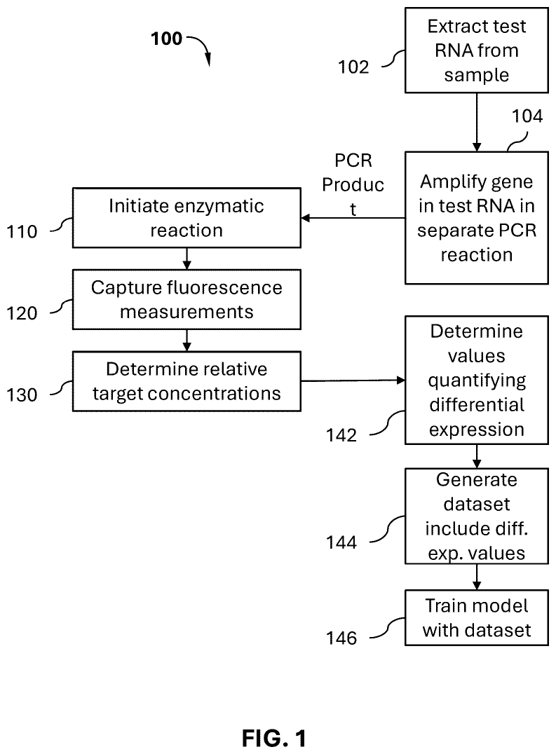

FIG. 1 illustrates a method 100 for multiplexed RNA quantification. The method 100 may enable high-throughput analysis of gene expression changes across numerous samples. In some cases, the method 100 may quantify over 4,500 gene-sample pairs in a single experiment.

The method 100 begins with RNA extraction at step 102. Extracted RNA may serve as input material for subsequent processing steps. In some cases, the method 100 may include amplifying a gene in the extracted RNA using a separate PCR reaction at step 104.

At step 110, the method 100 initiates an enzymatic reaction by mixing a reaction mixture. The reaction mixture may comprise a CRISPR effector protein, a target-specific crRNA, a fluorescent reporter molecule, T7 RNA polymerase, an input sample, and a reaction buffer. In some cases, the input sample may include PCR products from the separate PCR reaction performed in step 104.

The method 100 proceeds to step 120, where a plurality of fluorescence measurements are captured. Each fluorescence measurement may be captured at a different point in time while the reaction mixture is incubating. In some cases, fluorescence measurements may be captured using a microfluidic chip.

At step 130, the method 100 determines a plurality of relative target concentrations. This determination may involve fitting the plurality of fluorescence measurements to a mathematical model of the enzymatic reaction. The mathematical model may represent various aspects of the reaction kinetics.

The method 100 may further include determining values quantifying differential expression across treatment conditions at step 142. These values may be used to generate a dataset at step 144. In some cases, the method 100 may conclude with training a machine learning model using the generated dataset at step 146. The trained model may be used for various applications, such as predicting clinical or disease outcomes based on RNA quantification data.

By leveraging CRISPR-Cas technology and microfluidics, the method 100 may enable highly multiplexed and sensitive RNA quantification. This approach may facilitate large-scale gene expression studies and analyses that were previously challenging or impractical using traditional RNA quantification techniques.

The method 100 may begin with RNA extraction at step 102. In some cases, test RNA may be extracted from a sample. The RNA extraction step 102 may provide input material for subsequent processing steps in the method 100.

Following RNA extraction, the method 100 may proceed to step 104, where a gene in the test RNA is amplified in a separate PCR reaction. The amplification step 104 may increase the amount of target RNA sequences, potentially improving detection sensitivity in later steps of the method 100. In some cases, the input sample used in subsequent steps may include PCR products from this separate PCR reaction performed at step 104.

The method 100 may utilize RNase H2-dependent PCR (rhPCR) amplification prior to the Cas13 detection reaction. rhPCR may offer advantages for multiplexed amplification compared to conventional PCR techniques. In some cases, rhPCR may reduce primer dimer formation and improve amplification specificity when targeting multiple genes simultaneously.

The RNA extraction step 102 and amplification step 104 may serve as preparatory steps for the subsequent CRISPR-based detection and quantification process. These initial steps may help ensure sufficient target RNA is present and appropriately prepared for accurate detection and quantification in later stages of the method 100.

The method 100 proceeds to step 110, where an enzymatic reaction is initiated by mixing a reaction mixture. The reaction mixture may comprise several key components that enable RNA quantification.

In some cases, the reaction mixture may include a CRISPR effector protein. The CRISPR effector protein may be a Class 2 CRISPR effector protein. In some cases, the Class 2 CRISPR effector protein may be of Type V. In other cases, the Class 2 CRISPR effector protein may be of Type VI.

The CRISPR effector protein may comprise Cas12, Cas13, LwaCas13a (C2c2), LbCas12a (Cpf1), LbuCas13a, PsmCas13b, PspCas13b, CcaCas13b, AsCas12a, CeCas12a, PbCas12a, or a combination thereof. In some cases, the method 100 may use LwaCas13a as the CRISPR effector protein.

The reaction mixture may also include a target-specific crRNA. The crRNA may guide the CRISPR effector protein to bind to specific RNA sequences in the input sample.

A fluorescent reporter molecule may be another component of the reaction mixture. In some cases, the method 100 may include using a 6U FAM reporter as the fluorescent reporter molecule. The fluorescent reporter may generate a detectable signal when cleaved by activated CRISPR effector proteins.

T7 RNA polymerase may be included in the reaction mixture. T7 RNA polymerase may amplify target RNA sequences, potentially enhancing detection sensitivity.

The reaction mixture may further comprise an input sample. In some cases, the input sample may include PCR products from a separate PCR reaction performed in step 104. The separate PCR reaction may use rhPCR primers with a terminal blocking moiety in the form of rDDDDMx.

A reaction buffer may also be part of the reaction mixture. The reaction buffer may provide optimal conditions for the enzymatic activities of the CRISPR effector protein and T7 RNA polymerase.

By combining these components, the reaction mixture in step 110 may enable sequence-specific detection and quantification of RNA targets. The CRISPR effector protein, guided by the crRNA, may bind to and cleave target RNA sequences. This activity may lead to collateral cleavage of the fluorescent reporter molecules, generating detectable signals that correlate with target RNA concentration.

The method 100 proceeds to step 120, where a plurality of fluorescence measurements are captured. Each fluorescence measurement may be captured at a different point in time while the reaction mixture is incubating.

In some cases, capturing the plurality of fluorescence measurements may include capturing a fluorescence measurement every 1-15 minutes. For example, the method 100 may include capturing fluorescence measurements every 5 minutes. This time-resolved measurement approach may allow for tracking the progression of the enzymatic reaction over time.

Capturing the plurality of fluorescence measurements may include capturing fluorescence measurements while the reaction mixture is incubating at a predetermined temperature. In some cases, the method 100 may include incubating the reaction mixture at 37° C. Maintaining a consistent incubation temperature may help ensure reproducible reaction kinetics across different experiments.

The duration of fluorescence measurement capture may vary depending on the specific application. In some cases, capturing the plurality of fluorescence measurements may include capturing fluorescence measurements for 1-6 hours. This extended measurement period may allow for observing the full reaction progression, including initial rates and potential saturation effects.

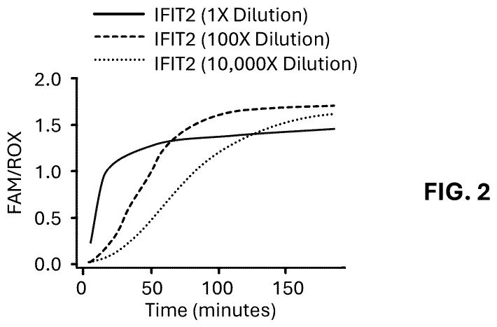

FIG. 2 illustrates example fluorescence kinetics data that may be obtained through the fluorescence measurement capture process of step 120. The graph shows fluorescence intensity over time for different sample dilutions. The solid line represents a 1× dilution, the dashed line represents a 100× dilution, and the dotted line represents a 10,000× dilution. These time-resolved measurements may reveal how the reaction kinetics vary with target concentration, potentially enabling more accurate quantification of RNA levels in the input sample.

The time-resolved nature of the fluorescence measurements captured in step 120 may be significant for accurate RNA quantification. By observing the full reaction progression rather than relying on a single endpoint measurement, the method 100 may capture important kinetic information. This kinetic data may allow for more robust fitting to mathematical models of the enzymatic reaction, potentially improving the accuracy and sensitivity of RNA quantification.

The method 100 may proceed to step 142, where one or more values quantifying differential expression across a plurality of treatment conditions are determined. These values may provide insights into how gene expression changes in response to various stimuli or experimental conditions.

In some cases, the differential expression analysis at step 142 may involve comparing RNA levels between treated samples and control samples. The method 100 may calculate fold changes in expression for each target RNA across different treatment conditions. For example, the method 100 may determine how the expression of interferon-stimulated genes changes in response to viral infection or interferon stimulation.

The differential expression analysis at step 142 may also enable the study of isoform switching kinetics during cellular processes such as epithelial-mesenchymal transition. The method 100 may quantify changes in the relative abundance of different RNA isoforms over time or across treatment conditions. This analysis may reveal shifts in splicing patterns or gene regulation during cellular transformations.

Following the differential expression analysis, the method 100 may proceed to step 144, where a dataset including the one or more values quantifying differential expression is generated. This dataset may compile the calculated expression changes for multiple genes across various samples and treatment conditions.

The dataset generated at step 144 may organize the differential expression values in a format suitable for further analysis or visualization. In some cases, the dataset may include metadata about the samples, treatment conditions, and experimental design. This comprehensive dataset may enable researchers to identify patterns or trends in gene expression changes across multiple experiments or conditions.

By generating a dataset of differential expression values at step 144, the method 100 may facilitate large-scale analyses of gene expression dynamics. The dataset may serve as input for various computational approaches, potentially revealing insights into complex biological processes or disease mechanisms.

The method 100 may conclude with step 146, where a machine learning model is trained using the dataset generated in step 144. The machine learning model may be configured to analyze patterns in RNA quantification data and make predictions about clinical or disease outcomes.

In some cases, the machine learning model may be a supervised learning algorithm, such as a random forest, support vector machine, or neural network. The dataset generated in step 144, which may include differential expression values across various treatment conditions, may serve as training data for the model.

The machine learning model may be trained to recognize patterns in gene expression profiles that correlate with specific clinical outcomes or disease states. For example, the model may learn to associate certain patterns of interferon-stimulated gene expression with increased susceptibility to viral infections.

In some cases, the trained machine learning model may be used to analyze new RNA quantification data from patient samples. The model may process the gene expression profiles and output predictions about potential disease risks or treatment responses.

The machine learning model may enable the translation of complex, high-dimensional RNA quantification data into actionable clinical insights. By leveraging the large-scale, multiplexed RNA quantification capabilities of the method 100, the model may be able to capture subtle patterns in gene expression that may be indicative of disease processes or treatment efficacy.

In some cases, the machine learning model may be continuously updated and refined as new RNA quantification data becomes available. This iterative training process may improve the model's predictive accuracy over time.

The integration of machine learning with the multiplexed RNA quantification approach of the method 100 may enable new applications in personalized medicine and disease prognosis. The ability to rapidly quantify expression levels of numerous genes across multiple samples, combined with machine learning-based analysis, may provide a powerful tool for understanding complex biological processes and making data-driven clinical decisions.

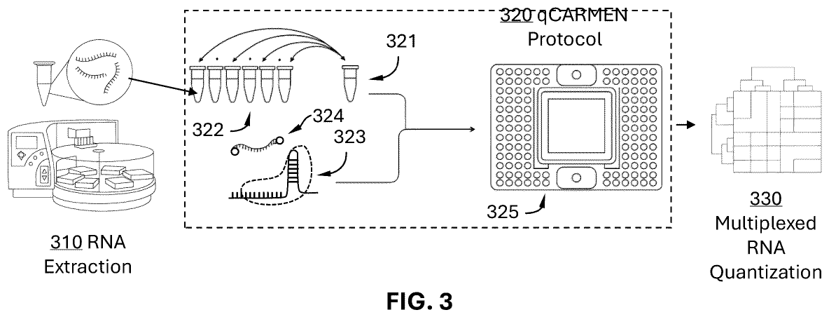

FIG. 3 illustrates a system diagram of an RNA quantification workflow. The system may include an RNA extraction module 310 that provides input material for subsequent processing steps.

The extracted RNA may feed into a qCARMEN protocol 320 which comprises multiple components arranged in series. The qCARMEN protocol 320 may enable simultaneous processing of 4,608 assay-sample pairs.

A primer pool 321 may be included as part of the qCARMEN protocol 320. In some cases, the primer pool 321 may comprise a plurality of RNase H2-dependent PCR (rhPCR) primers. The primer pool 321 may connect to a Pooled One Step RT-PCR module 322.

The output from the Pooled One Step RT-PCR module 322 may flow to a combination of Cas13+reporter mix 323 and Cas13 fluorescent reporter 324. In some cases, the Cas13+ reporter mix 323 may include a plurality of Cas13 detection reagents. The Cas13 fluorescent reporter 324 may generate detectable signals when cleaved by activated Cas13 proteins.

These components may interface with a microfluidic chip 325 where reactions take place. The microfluidic chip 325 may enable mixing of reaction components and simultaneous processing of multiple reaction mixtures. In some cases, at least 100 different reaction mixtures may be processed simultaneously on the microfluidic chip 325.

The qCARMEN protocol 320 may include additional components not explicitly shown in FIG. 3. For example, the protocol may include a plurality of crRNAs that guide Cas13 proteins to specific RNA targets. The protocol may also include a plurality of PCR reagents to support amplification reactions.

By combining these various components, the qCARMEN protocol 320 may enable highly multiplexed RNA quantification. The use of a microfluidic chip 325 for reaction processing may allow for miniaturization and parallelization of assays, potentially increasing throughput and reducing reagent consumption.

The system concludes with an RNA quantification module 330 that processes data from the microfluidic chip 325. The RNA quantification module 330 may receive fluorescence measurements generated through the interaction of components on the microfluidic chip 325.

In some cases, the RNA quantification module 330 may integrate the fluorescence measurements captured over time to determine relative target concentrations. The RNA quantification module 330 may fit the fluorescence data to a mathematical model of the enzymatic reaction occurring on the microfluidic chip 325.

The mathematical model used by the RNA quantification module 330 may comprise a system of differential equations representing individual reaction components. These components may include transcription, cis cleavage, and trans cleavage processes. By fitting the fluorescence data to this model, the RNA quantification module 330 may generate a single concentration-associated parameter for each reaction curve.

In some cases, the RNA quantification module 330 may process data from multiple reaction mixtures simultaneously. The module may be capable of analyzing fluorescence data from at least 100 different reaction mixtures processed on the microfluidic chip 325 in parallel.

The RNA quantification module 330 may normalize the calculated target concentrations to housekeeping gene concentrations. This normalization process may help account for variations in input RNA amounts across different samples.

In some cases, the RNA quantification module 330 may determine kinetic fold-changes in time-course experiments. The module may calculate relative changes in target concentrations from an initial timepoint. For infection studies or other experiments without multiple timepoints, the RNA quantification module 330 may compare normalized concentrations between treated and mock samples to determine changes in target expression.

The RNA quantification module 330 may output quantitative data on relative RNA levels across multiple targets and samples. This output may enable downstream analyses such as differential expression studies or machine learning model training.

By integrating fluorescence measurements and applying mathematical modeling, the RNA quantification module 330 may provide accurate and high-throughput RNA quantification results. This module may serve as the final step in translating raw fluorescence data from the microfluidic chip 325 into meaningful quantitative information about RNA levels in the input samples.

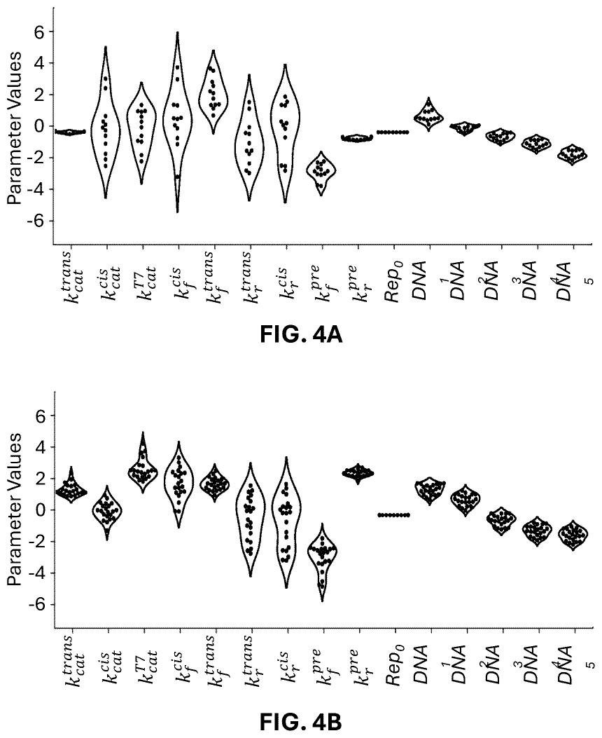

The mathematical model used for RNA quantification in the method may involve various parameters that describe the reaction kinetics. FIG. 4A and FIG. 4B illustrate parameter value distributions obtained from fitting the model to experimental data.

FIG. 4A shows violin plots representing the distribution of parameter values for one set of experiments, while FIG. 4B displays similar plots for a different set of experiments. Each violin-shaped distribution in these figures corresponds to a specific parameter in the mathematical model.

The width of each violin plot at different y-axis values indicates the frequency of occurrence for those parameter values. Wider sections of the plot suggest that those parameter values are more common, while narrower sections indicate less frequent values. The black dots within each violin shape may represent individual data points or measurements.

In some cases, the parameter distributions shown in FIG. 4A and FIG. 4B may be relatively consistent across different experiments or target genes. This consistency may suggest that the underlying reaction kinetics are similar for various RNA targets, potentially allowing for a generalized model to be applied across different quantification assays.

The spread of parameter values, as illustrated by the height of each violin plot, may provide information about the variability or uncertainty associated with each parameter. Parameters with narrow distributions may be more tightly constrained by the experimental data, while those with broader distributions may have greater uncertainty.

In some cases, certain parameters may show bimodal or multimodal distributions, as indicated by multiple wider sections in the violin plot. These patterns may suggest the presence of distinct subpopulations or different reaction regimes within the experimental data.

The parameter distributions shown in FIG. 4A and FIG. 4B may be used to assess the robustness and reliability of the mathematical model. Consistent and well-defined distributions may indicate that the model parameters can be reliably estimated from the experimental data, potentially leading to more accurate RNA quantification results.

By analyzing these parameter distributions, the method may be able to identify which aspects of the reaction kinetics are most variable or uncertain. This information may be used to refine the model or experimental protocols, potentially improving the accuracy and precision of RNA quantification.

In some cases, the parameter distributions may be used to generate confidence intervals or uncertainty estimates for the calculated RNA concentrations. By propagating the uncertainty in model parameters through the quantification process, the method may provide more comprehensive and statistically rigorous results.

The parameter value distributions illustrated in FIG. 4A and FIG. 4B may play a role in optimizing the RNA quantification method. By understanding which parameters are most variable or influential, the experimental design or data analysis procedures may be adjusted to focus on the most informative aspects of the reaction kinetics.

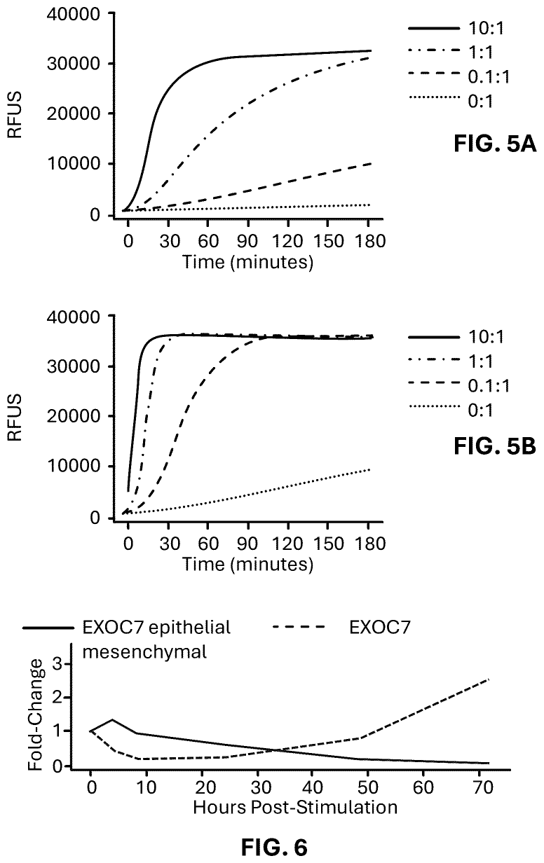

FIG. 5A and FIG. 5B illustrate fluorescence kinetics data for different sample ratios over time. These figures may provide insights into how the qCARMEN method detects and quantifies RNA targets at varying concentrations.

In FIG. 5A, the graph displays relative fluorescence units (RFU) plotted against time in minutes for four different sample ratios: 10:1, 1:1, 0.1:1, and 0:1. The curves demonstrate different rates of signal increase, with the 10:1 ratio reaching the highest RFU value of approximately 30,000 after about 60 minutes. This rapid increase in fluorescence for the highest concentration ratio may indicate efficient detection and amplification of the target RNA.

FIG. 5B shows similar RFU measurements over time for the same sample ratios, but with more rapid signal saturation. All ratios except 0:1 reach maximum RFU values of approximately 35,000 within the first 60 minutes of measurement. This faster saturation may suggest optimized reaction conditions or higher enzyme activities compared to the conditions used in FIG. 5A.

The differences in fluorescence kinetics between sample ratios may reflect the sensitivity of the qCARMEN method to varying RNA concentrations. Higher sample ratios, representing greater target RNA concentrations, typically show faster initial rates of fluorescence increase and reach higher maximum RFU values. This relationship between sample concentration and fluorescence kinetics may allow for quantitative analysis of RNA levels in unknown samples.

In some cases, the qCARMEN method may combine RNase H-dependent PCR (rhPCR) amplification and Cas13 detection using an assay module. The method may include performing RNase H2-dependent multiplexed amplification using a first predetermined concentration of MgCl₂ and a first predetermined enzyme activity of RNase H2 enzyme. The first predetermined concentration of MgCl₂ may be 1 mM to 5 mM. The first predetermined enzyme activity of RNase H2 enzyme may be 1 mU/μL to 5 mU/μL.

Following the RNase H2-dependent multiplexed amplification, the method may include detecting Cas13 using a Cas13 detection reaction. This Cas13 detection reaction may have a second predetermined concentration of MgCl₂, performed in series with the RNase H2-dependent multiplexed amplification. The second predetermined concentration of MgCl₂ may be 4 mM to 10 mM.

The combination of rhPCR amplification and Cas13 detection may contribute to the sensitivity and specificity of the qCARMEN method. The RNase H2-dependent amplification step may increase the amount of target RNA, while the subsequent Cas13 detection may provide sequence-specific signal amplification.

The fluorescence kinetics observed in FIG. 5A and FIG. 5B may reflect the combined effects of these amplification and detection steps. The initial rate of fluorescence increase may be influenced by the efficiency of the rhPCR amplification, while the maximum RFU values and signal saturation may be more closely related to the Cas13 detection reaction.

By analyzing the fluorescence kinetics for different sample ratios, as shown in FIG. 5A and FIG. 5B, the qCARMEN method may be able to quantify RNA levels across a range of concentrations. The distinct kinetic profiles for different sample ratios may allow for the development of calibration curves or mathematical models to relate fluorescence measurements to input RNA concentrations.

### Example 1

Gene expression levels are indispensable for understanding the functional states of biological systems and can explain the significance of certain genes across environmental conditions. Frequently, dozens, hundreds, or even thousands of genes will change in expression in response to environmental stimuli or genetic perturbations. The two standard methods for quantifying gene expression changes are quantitative reverse transcription polymerase chain reaction (RT-qPCR) and RNA sequencing (RNA-seq). While both methods are capable of probing differential gene expression, neither method is effective for quantifying dozens of genes across hundreds or thousands of samples. RNA-seq is too expensive for such high-throughput analyses, costing hundreds of dollars per sample. Conversely, RT-qPCR is too labor-intensive for highly multiplexed studies, and consumes large volumes of often precious samples. As a result, gene expression analyses typically involve an initial screen using RNA-seq and subsequent profiling of a few key genes across treatment conditions using RT-qPCR. A scalable, cost-effective method for quantifying RNA would allow us to study gene regulation at the pathway level with high temporal resolution, in response to multiple genetic or chemical perturbations.

An emerging paradigm of nucleic acid detection leverages the sequence-specific cis-and collateral trans-cleavage activities of type V and VI CRISPR-Cas systems for target detection. Recent work in viral diagnostics has shown that Leptotrichia wadei (Lwa) Cas13a, an RNA-targeting RNA nuclease, can be used in conjunction with RNA cleavage reporters to both detect and quantify viral targets such as SARS-COV-2 and influenza A virus. Cas13-based nucleic acid assays are sensitive, detecting targets at concentrations as low as one copy per microliter, and are more specific than nucleic acid hybridization. However, Cas13 detection reactions have continuous kinetics, unlike RT-qPCR, whose discrete cycles allow for differential cycle threshold (ACt) analysis. Thus, it has been challenging to infer input RNA concentrations from Cas13 detection kinetics to quantify gene expression in a highly multiplexed fashion.

To address this problem, the disclosed Cas13-based platform, sometimes called qCARMEN, enables high-throughput, multiplexed quantification of changes in host gene expression at a low cost and with minimal hands-on time. The qCARMEN platform uses a system of microfluidics, Cas13 detection reactions, and mathematical models to quantify target RNA concentrations in samples. The gene expression changes calculated using qCARMEN have a high degree of correlation with existing gold-standard methods and they can be calculated in a high-throughput, highly multiplexed fashion.

### Overview of the qCARMEN Workflow

The qCARMEN platform enables high-throughput, multiplexed RNA quantitation by parallelizing Cas13-based nucleic acid detection reactions and processing fluorescent readouts using a mathematical model of the reaction kinetics. A typical Cas13 detection reaction leverages the trans-cleavage activity of Cas13 to cleave fluorescent reporter molecules in the presence of target substrates. Prior work has demonstrated that these reactions can sensitively detect viral targets and that the rate of fluorescence saturation correlates with target concentration. However, CRISPR-based nucleic acid quantitation has proven to be challenging due to competition between amplicons and primer dimers during multiplexed amplification and the lack of a robust method for converting fluorescence kinetics to input nucleic acid concentrations. See FIG. 2. qCARMEN addresses these challenges by integrating a primer dimerization-resistant amplification strategy and reaction kinetics modeling using a system of differential equations.

The entire qCARMEN workflow requires less than two hours of total hands-on time and consists of three primary steps: complementary DNA (cDNA) synthesis, pre-amplification, and microfluidic chip loading. See FIG. 3. After cDNA synthesis, samples are pre-amplified in a multiplexed format using an RNase H2-dependent PCR (rhPCR) protocol to increase assay sensitivity and to incorporate a T7 promoter sequence onto target amplicons. in FIG. 3, a primer pool (321) is shown, along with the pooled one-step RT-PCR (322). rhPCR allows for significantly higher gene coverage in multiplexed amplification and eliminates primer dimers. PCR products are then loaded onto a microfluidic chip (325), where samples are combinatorially mixed with assay master mixes that utilize a target-specific CRISPR RNA (crRNA), LwaCas13a, and T7 RNA polymerase. In FIG. 3, a mixture including crRNA+enzyme complex (323) and a Cas13 fluorescent reporter (324) are shown. Upon mixing, reactions are incubated for three hours at 37° C. 37° C. with fluorescence measurements every 5 minutes. Raw fluorescence data are fit to a mathematical model to calculate relative target concentrations, which can be used to quantify differential expression across treatment conditions (see step 330, multiplexed RNA quantization).

Design of Primers and crRNAs

Primer and crRNA sequences for target transcripts were designed using an automated computational pipeline. The computational pipeline consists of three major components. First, crRNAs are designed for transcripts using ADAPT. Forward and reverse primer sequences are then designed for optimal crRNA designs based on G/C content, desired melting temperature, amplicon length, and the presence of monoand di-nucleotide repeats. Lastly, T7 promoter sequences are added to forward primers after both forward and reverse primer sequences are modified to support rhPCR amplification. All rhPCR primers and crRNAs were synthesized by Integrated DNA Technologies (IDT, Newark, NJ).

### Preparation of Synthetic Samples

Synthetic RNA samples were transcribed from gBlock DNA fragments (IDT, Newark, NJ). T7 promoter sequences included in the gBlock fragments and forward primers with T7 overhangs were used to generate templates of different sizes if needed. For in vitro transcription, the HISCRIBE® T7 High Yield RNA Synthesis Kit from New England Biolabs (NEB E2040S, Ipswich, MA) was used with the standard RNA synthesis protocol and incubated for 4 hours. After IVT was complete, IVT products were purified using a Monarch RNA Cleanup Kit (NEB, T2030L, Ipswich, MA) and stored at −80° C.

### RNA Extraction and Isolation

RNA was extracted from tissue culture cells using either the EZ-10 spin column kit (Bio Basic, Markham, Canada) or the MAGMAX™ MIRVANA™ Total RNA Isolation Kit (Thermo Fisher Scientific A27828, Waltham, MA). For EZ-10 column extractions, cells were detached at their respective timepoints and lysed in 450 μL of Buffer RLT after removal of supernatant. Cell lysates were then processed following the instructions in the EZ-10 manual. For timecourse experiments involving more than 48 samples, the MAGMAX™ MIRVANA™ Total RNA Isolation Kit was used in conjunction with the KingFisher extraction platform. After each timepoint, cells were immediately lysed in MAGMAX™ Lysis/Binding Solution Concentrate (Thermo Fisher Scientific AM8500, Waltham, MA) and left in storage at −20° C. until all timepoints were collected and ready for automated extraction. RNA was eluted in 50 μL of water and further processed with TURBO™ DNase (Thermo Fisher AM2238, Waltham, MA). RNA samples were then purified using RNAClean XP (Beckman Coulter A63987, Brea, CA) SPRI beads and eluted in 22 μL of water. Purified RNA extracts were then aliquoted and stored at −80° C. until ready for usage.

### Generation of Virus Stocks

Hepatitis C and YFV17D: HCV RNA and subsequent viral stocks were produced as previously described 29. In brief, viral RNA was produced via in vitro transcription of an XbaI-linearized Jc1 (p7nsGluc2A) plasmid using the T7 RiboMAX Express Large Scale RNA Production kit (Promega P1320) as outlined in the user manual 30. Viral RNA was purified using the Qiagen RNeasy Mini Kit (Qiagen 74104) following manufacturer's instructions, and quality control was performed by gel electrophoresis to ensure no significant RNA degradation. Viral RNA stocks were stored as 5 μ galiquots at −80° C. RNA was electroporated into Huh7.5.1 cells The pellet was resuspended in the appropriate volume of cold DPBS to achieve a concentration of 1.5 E 7 cells/mL. 6E6 cells were then electroporated in a 2 mm path length electroporation cuvette (BTX Harvard Apparatus; Holliston, MA) with 5 μg of viral RNA using an ECM 830 Square Wave Electroporation System (BTX) at the following settings: five pulses, 99 μs per pulse, 1.1 s pulse intervals, 860 V. Following a ten-minute incubation at room temperature, the electroporated cells were seeded into p150s and maintained in 5% FBS DMEM. Media was changed one day postelectroporation, and supernatants were collected daily for six days and stored at 4° C. The pooled supernatants were passed through a 0.22 μm vacuum filter and subsequently concentrated to ˜100 mL in an EMD Millipore Stirred Cell (Cole-Parmer). The TCID50/mL (Reed and Muench, 1938) of concentrated virus was determined after one freeze-thaw by limiting dilution assay.

### Quantification of Infection

Cells were pelleted, fixed with 4% paraformaldehyde (PFA) (Sigma-Aldrich) and permeabilized in 0.1% (w/v) Saponin and 1% (v/v) FBS in DPBS. Pellets were subsequently incubated for 1 hour at room temperature with anti-E (diluted 1:100, 4G2, Novus) for flavivirus-infected cells and murine-produced Clone 9E10 primary antibody specific for NS5A (kindly provided by Dr. Charles Rice at The Rockefeller University) diluted 1:8000 in FACS buffer (1% FBS (v/v) in DPBS) for HCV29,32. Cells were then washed with DPBS and incubated at 4° C. for 30 minutes in the dark with secondary antibody (diluted 1:250, Invitrogen A-21235, Waltham, MA) conjugated with FITC (flaviviruses) or Alexa 647 (HCV). Cells were subsequently pelleted, washed once with FACS buffer (1% FBS in DPBS) and then analyzed in FACS buffer on a BD LSRII flow cytometer (BD Biosciences, Franklin Lakes, N). Flow cytometry data were processed in FlowJo Software version 10.4.2 (FlowJo, Becton Dickson, Franklin Lake, NJ).

cDNA Synthesis

After RNA extraction and isolation, samples were converted to cDNA using SuperScript IV (Thermo Fisher Scientific 18090010, Waltham, MA) in 20 μL reactions. First, 5 μL of template RNA at 20-200 ng/μL were added to 6 μL of water, 1 μL of 50 mM oligo (dT) 20 (Thermo Fisher Scientific 18418020, Waltham, MA), and 1 μL of 10 mM dNTP mix (NEB N0447S, Ipswich, MA). This initial premix was heated at 65° C. for 5 minutes, then incubated on ice for 1 minute. A pre-mix second consisting of 4 μL of 5×SSIV buffer, 1 μL of 100 mMDTT, 1 μL of murine RNase inhibitor (NEB M0314S), 0.5 μL of SuperScript IV (Thermo Fisher Scientific 18090010, Waltham, MA), and 0.5 μL of water was prepared. After incubation on ice of the first master mix was complete, the second pre-mix (7 μL in total) was added to the 13 μL of the first mix, creating a 20 μL reaction volume. The final reaction mixture was incubated at 55° C. for 20 minutes and inactivated at 80° C. for 10 minutes.

### RNase H2-Dependent POR Amplification

RNA samples were pre-amplified prior to Cas13 detection experiments by adding 2 μL of input material to a 25 μ Lreaction consisting of AmpliTaq DNA polymerase (Thermo Fisher Scientific N8080152, Waltham, MA), RNase H2 enzyme (IDT 11-03-02-02, Newark, NJ), and GENI rhPCR primers. GEN1 rhPCR primers consist of the conventional primer sequence with a terminal blocking moiety that takes the form of rDDDDMx, where D represents a DNA base, r represents the RNA base, M represents a mismatched DNA base, and x represents the blocker.

### Assay and Sample Mix Preparation

Assay mixes were prepared in 16 μL volumes for each unique crRNA target consisting of 1 μL of 50 U/μL NxGen T7 Polymerase (Biosearch Technologies 30223-1), 2 μL of 800 nM LwaCas13a (Genscript), 1 μL of a 1.6 μM target-specific Cas13 crRNA, 8 μL of 2× Fluidigm Assay Loading Buffer, and 4 μL of nuclease-free water. 1.5× sample premixes were created with 2.4 μL of 10×T7 Buffer (Biosearch Technologies F88905-1, Hoddesdon, United Kingdom), 3.2 μL of 7.5× sample buffer, 0.2 μL of murine RNase inhibitor (NEB M0314S, Ipswich, MA), 0.96 μL of 25 mM rNTP mix (NEB N0466S, Ipswich, MA), 0.12 μL of 100 μM 6U FAM reporter (IDT), 0.48 μL of 50×ROX reference dye (Thermo Fisher Scientific 12223012, Waltham, MA), 1.20 μL of 20×GE buffer, and 7.44 μL of nuclease-free water. 7.5× Sample Buffer solutions were made in 5 mL aliquots with 375 μL of Tris-HCl, 75 μL of 5 MNaCl, 37.5 μL of 1 MMgCl2, 750 mg of PEG-8000, and 4512.5 μL of nuclease-free water. Sample premixes were added to pre-amplified sample mixtures in a 2:1 ratio of premix to sample.

### Integrated Fluidic Circuit (IFC) Chip Loading

192.24 IFCs (Standard BioTools 100-6266, South San Francisco, CA) were used for microfluidic mixing of assay and sample mixes. 3.5 μL of each sample mix were loaded into each IFC sample well and 3.5 μL of each assay mix were loaded into IFC assay wells. Remaining empty IFC wells were filled with either an assay or sample filler mix. Assay filler mix consisted of assay loading buffer and water in a 1:1 ratio and sample filler mixes consisted of nuclease-free water, 20×GE buffer, and ROX reference dye in a 93:5:2 ratio. Actuation and pressure fluid (Standard BioTools 100-6267, South San Francisco, CA) were added to the chip. 192.24 IFCs were then primed and loaded in the Fluidigm HX Controller using the default prime and load script for 30 minutes. After priming, IFCs were loaded immediately into the Biomark HD (Standard BioTools, South San Francisco, CA) to initiate the reaction. Reactions were ru. B,n for 3 hours at 37° C. with 37 imaging steps on the fluorescein amedite (FAM) channel (excitation at 494 nm, emission at 518 nm) separated in 5-minute intervals.

### Model Fitting and Data Analysis

Parameters were generated for the 11-parameter Cas13 reaction model by fitting the model on fluorescence kinetics data from dilutions of RNA extracted from YFV17D-infected and IFNstimulated cells. Sets of random parameters were generated and used to fit the model on dilution curves for multiple genes. A “shared fitting” approach was used to find fixed model parameters that were shared between dilutions of a given RNA target with only the input concentration parameter across dilutions. Parameter sets that successfully fit real-world fluorescence data were collected and averaged across dilutions and targets to create an initial parameter set for future model fits and analyses.

To determine input concentrations from experimental data, the initial parameter set was used to generate fits to fluorescence data using the qCARMEN model. Input concentrations were determined for targets of interest as well as two housekeeping genes, GAPDH and HPRT1. Input concentrations for all samples were normalized to housekeeping gene concentrations. Normalized concentrations were used to determine kinetic fold-changes in time-course experiments by determining relative changes from the initial timepoint. For infection data, where multiple timepoints were not collected, normalized concentrations for treated samples were compared to normalized concentrations for mock samples to determine changes in target expression.

### Example 2 (Validation of Cas13—Based Transcript Detection as Highly Sensitive and Specific)

To validate the sensitivity of qCARMEN, synthetic transcripts of the housekeeping genes glyceraldehyde 3-phosphate dehydrogenase (GAPDH) and hypoxanthine phosphoribosyltransferase 1 (HPRT1) were generated via in vitro transcription (IVT). IVT products were DNasetreated and column-purified prior to normalizing transcript concentrations to 1012 copies/μL. Synthetic housekeeping gene samples were serially diluted by a factor of ten to generate transcript samples with concentrations ranging from 1 to 108 copies/μL. To establish the limit of detection of the qCARMEN reaction for each housekeeping gene, a no-template control (NTC) was used consisting of only nuclease-free water as input to two reactions separately aiming to detect either GAPDH or HPRT1. The serial dilutions were used as inputs for qCARMEN reactions detecting GAPDH and HPRT1 and fluorescence outputs were collected. Fluorescence data were compared to the NTC for each gene to establish a comparative limit of detection. Based on the NTC outputs, limits of detection for both GAPDH and HPRT1 were determined to be approximately 100-1,000 copies/μL and 1-10 copies/μL, respectively.

To test the specificity of the qCARMEN workflow, total RNA was extracted from human Huh7.5 hepatoma cells and purified after DNase I treatment. Eight genes were then individually amplified in the extracted RNA across eight separate PCR reactions. Each of the eight PCR products was then combined with eight unique assay mixes each containing a crRNA targeting one of the eight target genes for a total of 64 assay-sample mix combinations. Cas13-mediated detection of target genes was highly specific, generating a positive signal approximately 15 times above background.

### Example 3—Modeling the Cas13 Detection Reaction for Effective Transcript Quantitation

To quantify changes in gene expression using fluorescence data from Cas13 detection reactions, fluorescence curves need to be converted into scalar values that correlate with input RNA concentrations. Prior work has inferred input RNA concentrations by calculating half-maximal inhibitory concentration (IC₅₀) values from fluorescence kinetic curves; however, this requires saturating reaction kinetics and therefore severely limits the dynamic range of gene expression quantitation. To circumvent this limitation, the Cas13 detection system was modeled using a system of differential equations representing individual reaction components including transcription, cis cleavage, and trans cleavage/

Specifically, to better infer concentration values from fluorescence data, the Cas13 detection reaction was mathematically modeled with a system of differential equations. The model accounts for four primary processes: target transcription, Cas13-crRNA complexing, cis-cleavage, and trans-cleavage. While the complete reaction is certainly more complex, it was found that this simplified description of the overall reaction is capable of modeling a wide range of experimentally generated fluorescence curves and inferring concentration values that correlate well with gold-standard method.

The simplified description is as follows:

$$\begin{matrix}DNA + T 7 {\to }^{{k}_{cat}^{T 7}} DNA + T 7 + Target \\ C 13 + crRNA + T \begin{matrix}{\to }^{{k}_{f}^{pre}} \\ {←}_{{k}_{r}^{pre}}\end{matrix} C 13 - crRNA \\ C 13 + crRNA + T \begin{matrix}{\to }^{{k}_{f}^{cis}} \\ {←}_{{k}_{r}^{cis}}\end{matrix} C 13 - crRNA - T {\to }^{{k}_{cat}^{cis}} AE \\ AE + FR \begin{matrix}{\to }^{{k}_{f}^{trans}} \\ {←}_{{k}_{r}^{trans}}\end{matrix} AE - FR {\to }^{{k}_{cat}^{trans}} AE + F\end{matrix}$$

Parameters generated for the model were largely consistent across different targets, exhibited linearity in predicted concentration trends, and were able to fit distinct types of fluorescence kinetics (see, e.g., FIGS. 4A-4B). Use of the model enables the ability to create precise fits to the fluorescence data and generate a single concentration-associated parameter for each curve.

With the derived model parameters, it was aimed to quantify changes accurately and precisely in gene expression. To validate the model, a dilution series of synthetic RNA targets were first generated, and the qCARMEN assay was used to generate fluorescence curves for each dilution. Then, by fitting the model to the fluorescence data, changes in input RNA levels were calculated across these dilutions and compared the calculated change in input RNA levels to the dilution factor. By plotting the logarithm of calculated “fold-change” values against a one-to-one line that goes through the origin, a correlation coefficient of 0.88 was calculated, and therefore it was found that the model generates concentration values that can be used to accurately quantify changes in gene expression.

Next, the model was validated in a cellular context by infecting Huh7 cells with the yellow fever virus (YFV) vaccine strain 17D (YFV17D) and observing changes in expression across 10 genes selected from an RNA-Seq analysis of Huh7 cells harboring subgenomic replicons (SGRs) of YFV17D or the virulent YFV-Asibi parental strain.

After extracting RNA from infected samples and mock cells, both RT-qPCR and qCARMEN were used to independently determine expression changes. By plotting the logz fold-changes from the two methods against each other, a high one-to-one linearity was found between qPCR and qCARMEN results with a correlation coefficient of 0.90. The results from these two experiments demonstrate that qCARMEN can accurately quantify gene expression changes in consensus with qPCR.

### Example 4—Analysis of IFN Response Kinetics Using the qCARMEN ISG Panel

The interferon (IFN) response plays a key role in fighting infection and studying ISGs can provide mechanistic insights into pathogen defense pathways. IFN-mediated cellular responses to infections have been studied in great detail and involve hundreds of interferon-stimulated genes (ISGs) and thus lend themselves as a test case for qCARMEN. Examples of well-characterized ISGs include the oligoadenylate synthetase (OAS) family, which is responsible for detecting foreign RNA, and the IFN-induced transmembrane (IFITM) proteins, which typically inhibit viral endocytic fusion events. Thus, it was sought to design a qCARMEN panel covering approximately two dozen commonly induced ISGs.

The ISG panel was first applied towards studying differences in ISG dysregulation in response to type I and III IFN stimulation. While both type I and III IFNs can induce antiviral states, the two types generate distinct ISG response profiles. Type I IFN stimulation typically triggers a fast, but robust ISG response whereas type III IFN stimulation induces weaker, but longer lasting responses. Human bone osteosarcoma epithelial U2OS cells, which bear both type I and III interferon receptors, were stimulated with IFNs β, α2, and λ3 (referred to as IFNs β, α, and A throughout the rest of this text) at doses that previously determined to generate strong ISG responses. IFN-stimulated and mock cells were taken down over the course of 72 hours, after which RNA was extracted and quantified using qCARMEN for all ISGs that exhibited baseline expression in mock cells.

More specifically, for this example, naïve U2OS cells were cultured in 24-well cell culture plates and grown to 80% confluency prior to interferon stimulation. Cells were then stimulated with 120IU/mL of IFN-β, 800 IU/mL of IFNα, and 40 ng/ml of IFN-λ. Stimulated cells were then incubated at 37° C. for 72 hours and timepoint samples were collected for IFN-stimulated and mock wells at 0, 1, 2, 4, 8, 24, 48, and 72-hour timepoints. At each timepoint, cells were immediately lysed in MAGMAX™ Lysis/Binding Solution Concentrate (Thermo Fisher Scientific AM8500, Waltham, MA) and stored at −20° C. until all timepoint samples were ready for RNA extraction. Extractions were performed using the MAGMAXTM MIRVANATM kit (Thermo Fisher Scientific, Waltham, MA) and automated using the KingFisher extraction system. Extracts were treated with TURBOTM DNase and further purified using RNAClean XP (SPRI) beads. Final RNA extracts were then aliquoted and stored at −80° C. until required.

By using qCARMEN to quantify changes in gene expression in the IFN-stimulated cells relative to mock samples, it was found that most ISGs were upregulated across type I and III IFN-stimulated cells as expected based on prior results and publicly available data on IFN-mediated ISG dysregulation. Additionally, type I and III IFN stimulation typically induced the same direction of ISG dysregulation for most genes in the panel after 24 hours. However, for two ISGs—moloney leukemia virus 10 like-1 (MOV10) and single stranded DNA binding protein 3 (SSBP3)—different directionality in ISG changes were observed across the IFN subtypes.

Plotting the distribution of fold-change values at each time point shows the differences in kinetics and response strength for each IFN. Stimulation with IFN-β, a type I IFN, generates a rapid ISG response that peaks at 4 hours. Overall, IFN-λ induces a slower ISG response that peaks at 24 hours—much later than with IFN-β—which is consistent with previous observations.

The data from the qCARMEN panel also allows for a more in-depth kinetic analysis of specific genes. As previously mentioned, type I IFN responses can be characterized by swift and marked changes in expression. However, this generalization does not necessarily apply to all downstream ISGs. By specifically looking at the kinetic profiles of BST2, OASL, and SSBP3 during IFN-β stimulation, distinct examples of up-regulation (BST2), down-regulation (SSBP3), and delayed dysregulation (OASL) were observed all in response to stimulation by the same type I IFN.

The throughput and multiplexing capabilities of qCARMEN also allow us to calculate pairwise correlations across genes to assess co-regulation within different interferon responses. As expected, when calculating Spearman correlations for genes responding to IFN stimulation, it was found that most ISGs are positively correlated with one another. However, some genes—SSBP3, ADAR, DDIT4, MOV10, and IRF7-exhibit negative correlations in cells stimulated with IFN-β. Similarly, IFN-α and IFN-A stimulation result in high positive correlations for most ISGs, though ADAR and SSBP3 are also negatively correlated in the IFN-α and λ datasets.

### Example 5—Application of ISG Panel Towards Characterizing Host Responses to Flavivirus Infections

Flaviviruses are a family of positive-sense, single-stranded RNA viruses that cause infections in over 500 million people annually. Within this family of viruses, infections with different viruses can sometimes cause vastly different clinical outcomes. To demonstrate the broad applicability of the disclosed techniques, and specifically the ISG panel of Example 4, and to gain insights into the differences in cell intrinsic innate immune responses, Huh7 hepatoma cells were infected with an array of flaviviruses to characterize the host responses in cell culture. Huh7 cells were infected with dengue serotypes (DENV)-1, DENV-2, DENV-3, DENV-4, Langat (LGTV), Usutu (USUV), Zika (ZIKV), hepatitis C viruses (HCV), or the YFV17D strain. 2-5 days post infection, the frequencies of viral antigenbearing cells were confirmed via flow cytometry and cells were lysed to extract total RNA in bulk from infected and uninfected cells in each treatment well. RNA from uninfected Huh7 cells was used to generate dilution series for quantitation. Additionally, to ensure that dysregulated genes that may not be present in high concentrations in uninfected cells were represented in the dilution series, total RNA from DENV-4-infected cells was used to generate another dilution series more representative of the infected state.

More specifically, for this example, 24 hours prior to infection, Huh7 cells were seeded in a 6-well format at densities of 1.5E5 (for HCV) or 6e5 cells (for all other viruses) per well. Infections were conducted in triplicate wells at an MOI of 0.1 (HCV, DENVs, ZIKV, LGTV, USUV) or 0.05 (YFV-17D). Viral inoculum was applied for 8 hours (HCV) or 2 hours (all of the other viruses) after which the wells were washed once with DMEM and the media changed to 10% (v/v) FBS 1% (v/v) P/S DMEM. Cells were trypsinized at 2 days post-infection (dpi) (YFV-17D), 3 dpi (DENV-1, DENV-4, ZIKV, USUV, LGTV) or 5 dpi (HCV, DENV2, DENV3) and split into two tubes for viral antigen staining and pelleted for RNA was extracted using the EZ-10 Spin Column Total RNA Miniprep Super Kit (Bio Basic).

The ISG panel shows distinct ISG responses across the different flavivirus infections both in the magnitude and direction of changes as well as the sets of dysregulated genes. By looking at the distribution of calculated fold-changes across viruses for each gene, it could be seen that five ISGs in particular—DNA damage inducible transcript 4 (DDIT4), SSBP3, myxoma resistance protein 1 (MX1), OASL, and ISG15—exhibit the greatest variance across the array of flaviviruses. DDIT4 seemed to decrease in expression for most genes, particularly for HCV and ZIKV. The latter four highly dysregulated ISGs all generally demonstrated an increase in expression with up to four orders of magnitude in variation between viruses. The remaining ISGs in the panel deviated minimally from baseline expression levels for the majority of viruses.

To better uncover key genes relevant in the immune response to flavivirus infections, Spearman correlations were performed between genes based on the gene signature vectors generated for each infection. The Spearman correlation matrix shows two primary clusters of positively correlated genes. Notably, SSBP3 exhibited strong inverse correlations with the other ISGs on the panel except for Moloney leukemia virus 10 protein (MOV10) and three prime repair exonuclease 1 (TREX1). Notably, SSBP3 and MOV10 both exhibited similar negative correlations with other ISGs when stimulated with type I IFNs β and α. Not much is known about the role that SSBP3 plays in the antiviral response, but it is known that SSBP3 is a transcriptional regulator involved in interfering with viral translation. One possible explanation for this inverse correlation is that SSBP3 plays a role in regulating the IFN response by suppressing ISG expression to avoid an overly extended immune response. Because MOV10 and TREX1 do not exhibit significant changes in expression in response to any of the viral infections, it makes sense that SSBP3 is not inversely correlated with these two ISGs.

Lastly, the ISG responses for each flavivirus infection were compared to the ISG profiles induced by type I and III IFN stimulation. To do so, PCA analysis was performed on the gene expression signatures for each IFN and timepoint. Samples were collected at those when the frequency of viral antigen-bearing cells reached peak (80-100%) levels. Since the timepoints at which infected cells were taken down across the different viruses, incorporating gene expression data from each timepoint allows inclusion of a temporal component in the characterization of each flavivirus. Plotting each gene expression vector allows one to first observe overlapping trajectories in the progression of the ISG responses. IFNs a and A exhibit very similar trajectories during the first four hours post-infection, diverge at the 8-hour timepoint, and eventually converge after 72 hours. IFN-β exhibits a completely different trajectory altogether. The host responses of 8/10 viruses lie far from the unperturbed 0-hour timepoint. Calculating the Euclidean distance between flavivirus ISG profiles and IFN stimulation timepoints using generated PCA components provides a quantitative description of the trajectory analysis. Most viruses used here-DENV-1, DENV-2, DENV-3, DENV-4, LGTV, USUV, and YFV17D—have ISG signatures that most closely resemble IFN α or λ responses during the first 1-4 hours of stimulation. The remaining flaviviruses-HCV and ZIKV—lie closer to earlier timepoints for IFN β. Collectively, these data demonstrate that qCARMEN is readily suitable to characterize complex transcriptional changes induced by cytokines and viral infection.

### Example 6—Quantitation of Alternative Splicing in Breast Cancer Cells

An additional layer of complexity in host transcriptional changes arises from alternative splicing of mRNAs. While quantifying changes in splice isoforms is possible with existing RT-qPCR techniques, designing primer sets that specifically amplify one particular isoform can be challenging due to primer location and design constraints. Because Cas13-based RNA targeting is mediated by both the crRNA and the Cas13 enzyme, Cas13 crRNAs are generally more robust than conventional RT-qPCR probes as secondary structure, GC content, and annealing temperature are less likely to influence specificity and target binding. Thus, qCARMEN stands to offer an effective alternative to RT-qPCR for measuring changes not only across genes, but also across distinct splice isoforms.

To validate qCARMEN for isoform quantitation, we studied isoform switching events in breast cancer cell lines during epithelial-mesenchymal transition (EMT). Studies indicate that EMT plays a meaningful role in tumor progression due to the loss of cell adhesiveness and activation of motility during EMT. Additionally, alternative splicing has also been shown to occur during EMT in numerous cancer cell lines. EMT can easily be induced in such cell lines by treatment with recombinant transforming growth factor β (TGF-β), allowing the ability to generate samples with differentially expressed splice isoforms.

A qCARMEN panel was designed to observe alternative splicing for CD44, fibroblast growth factor (FGFR1), mothers against decapentaplegic 2 (SMAD2), and exocyst complex component 7 (EXOC7)-genes that have been previously characterized in the context of alternative splicing during EMT. MCF7 and T47D breast cancer cell lines were treated with 5 ng/μL of TGF-β and samples were collected over the course of 72 hours. Extracted RNA was used as input for the qCARMEN workflow.

More specifically, MCF7 and T47D cell lines were perturbed using 5 ng/μL of recombinant human TGF-β (R&D Systems 240-B-002/CF, Minneapolis, MN) over the course of 3 days. 12 hours prior to treatment with TGF-β, cells were serum-starved using DMEM supplemented with 1% (v/v) FBS and 1% (v/v) P/S to improve uptake of TGF-β. After serum starvation, cells were treated with 5 ng/ml of recombinant TGF-β over 72 hours and timepoints were collected at 0, 1, 2, 4, 8, 24, 48, and 72 hours. RNA extractions were performed using the same MAGMAX™ protocol as described in the IFN stimulation example.

To first confirm that Cas13 is capable of distinguishing between splice isoforms, synthetic isoforms were generated for FGFR2 and crRNAs and primer pairs targeting either the epithelial or the mesenchymal isoform were designed. To assess specificity, isoform-specific assay mixes were combined with both the synthetic epithelial and mesenchymal target mixes to generate a fluorescent readout over the course of three hours. Detection was largely specific for both isoforms with some background detection of the FGFR2 IIIc isoform in the IIIb target mix. Dilutions of each isoform were then created with the other isoform present in solution a constant concentration across each dilution. Cas13 detection assays generated distinct fluorescence curves for each dilution for both the IIIb and IIIc isoforms of FGFR2, demonstrating that qCARMEN can distinguish different concentrations of splice isoforms and quantify changes in isoform expression. See FIGS. 5A-5B.

Next, qCARMEN was applied towards the analysis of differentially expressed splice isoforms in MCF7 and T47D cells after induction of EMT. The genes in the panel showed the expected increases in expression across the mesenchymal forms relative to the epithelial forms over the course of three days for both cell lines. The changes in gene expression were more noticeable in T47D cells, especially for the mesenchymal splice isoform of EXOC7, which exhibited nearly a 6-fold increase in expression between the 0-and 72-hour timepoints. CD44, a cell surface protein known to undergo alternative splicing during EMT, exhibited an increase in its mesenchymal form and a decrease in its epithelial isoform after 72 hours of TGF-β treatment as expected from past studies.

In addition to assessing long-term fold-changes in expression, the kinetics of splice isoform expression patterns was examined and striking behaviors in the dynamics of the epithelial and mesenchymal isoforms were identified. In particular, during EMT in T47D cells, the epithelial and mesenchymal isoforms of EXOC7 do not exhibit a simplistic, immediate decrease or increase in the dominant isoform. See FIG. 6. During the first two hours of TGF-β stimulation, the mesenchymal EXOC7 isoform decreases relative to the initial timepoint and the epithelial form increases until the 4-hour time point. After reaching these critical points, the epithelial and mesenchymal forms begin to trend towards the expected direction of change, and by the 72-hour time point, an increase is seen in the mesenchymal form and a decrease is seen in the epithelial form. Taken together, these results establish proof-of-concept for the utility of qCARMEN to monitor the complex dynamics of different RNA splice forms during EMT transitions.

A number of implementations have been described. Nevertheless, it will be understood that various modifications may be made without departing from the spirit and scope of the disclosure. Accordingly, other implementations are within the scope of the following claims.

1. A method for multiplexed RNA quantification, comprising:
initiating an enzymatic reaction by mixing a reaction mixture, the reaction mixture comprising a CRISPR effector protein, a target-specific crRNA, a fluorescent reporter molecule, T7 RNA polymerase, an input sample, and a reaction buffer;
capturing a plurality of fluorescence measurements, each fluorescence measurement captured at a different point in time; and
determining a plurality of relative target concentrations by fitting the plurality of fluorescence measurements to a mathematical model of the enzymatic reaction.

2. The method of claim 1, wherein said CRISPR effector protein is a Class 2 CRISPR effector protein.

3. The method of claim 2, wherein said Class 2 CRISPR effector protein is of Type V.

4. The method of claim 2, wherein said Class 2 CRISPR effector protein is of Type VI.

5. The method of claim 2, wherein said CRISPR effector protein comprises Cas12, Cas13, LwaCas13a (C2c2), LbCas12a (Cpf1), LbuCas13a, PsmCas13b, PspCas13b, CcaCas13b, AsCas12a, CeCas12a, PbCas12a, or a combination thereof.

6. The method of claim 1, further comprising determining one or more values quantifying a differential expression across a plurality of treatment conditions.

7. The method of claim 6, further comprising generating a dataset including the one or more values.

8. The method of claim 7, further comprising training a machine learning model using the dataset.

9. The method of claim 8, wherein the machine learning model is configured to predict a clinical outcome or a disease outcome.

10. The method of claim 1, wherein the mathematical model comprises a system of differential equations representing individual reaction components including transcription, cis cleavage, and trans cleavage.

11. The method of claim 1, wherein fitting the plurality of fluorescence measurements includes generating a single concentration-associated parameter for each curve.

12. The method of claim 1, wherein the mixing occurs on a microfluidic chip.

13. The method of claim 1, wherein capturing the plurality of fluorescence measurements includes capturing a fluorescence measurement every 1-15 minutes.

14. The method of claim 1, wherein capturing the plurality of fluorescence measurements includes capturing fluorescence measurements for 1-6 hours.

15. The method of claim 1, wherein capturing the plurality of fluorescence measurements includes capturing fluorescence measurements while the reaction mixture is incubating at a predetermined temperature.

16. The method of claim 1, further comprising extracting test RNA from a sample.

17. The method of claim 16, further comprising amplifying a gene in the test RNA in a separate PCR reaction, where the input sample includes a PCR product from the separate PCR reaction.

18. The method of claim 1, wherein the reaction mixture consists of the CRISPR effector protein, the target-specific crRNA, the fluorescent reporter molecule, T7 RNA polymerase, the input sample, and the reaction buffer.

19. The method of claim 1, wherein a plurality of different reaction mixtures are processed simultaneously.

20. The method of claim 19, wherein at least 100 different reaction mixtures are processed simultaneously.

21. An assay module for RNA quantification, comprising:
a RNase H2-dependent PCR (rhPCR) primer;
a crRNA;
a Cas13 detection reagent; and a PCR reagent.

22. The assay module of claim 21, wherein:
the RNase H2-dependent PCR (rhPCR) primer comprises a plurality of RNase H2-dependent PCR (rhPCR) primers;
the crRNA comprises a plurality of crRNA;
the Cas13 detection reagent comprises a plurality of Cas13 detection reagents; and/or
the PCR reagent comprises a plurality of PCR reagents.

23. A method for combining RNase H-dependent PCR (rhPCR) amplification and Cas13 detection, comprising:
providing an assay module of claim 21;
performing RNase H2-dependent multiplexed amplification using a first predetermined concentration of MgCl₂ and a first predetermined enzyme activity of RNase H2 enzyme; and in series with the RNase H2-dependent multiplexed amplification, detecting Cas13 using a Cas13 detection reaction having a second predetermined concentration of MgCl₂.

24. The method of claim 23, wherein the first predetermined concentration is 1 mM to 5 mM, the first predetermined enzyme activity is 1 mU/μL to 5 mU/μL, and the second predetermined concentration is 4 mM to 10 mM.
